(19) United States
(12) Patent Application Publication
(10) Pub. No.: US 2023/0143352 A1
(43) Pub. Date: May 11, 2023

(54) GENISTEIN TREATMENT OF INFLAMMATORY PULMONARY INJURY

(71) Applicant: Humanetics Corporation, Minneapolis, MN (US)

(72) Inventors: Artur A. Serebrenik, Minnetonka, MN (US); Michael D. Kaytor, Maplewood, MN (US); John C. Dykstra, Edina, MN (US); Ronald J. Zenk, Shorewood, MN (US)

(21) Appl. No.: 17/917,316

(22) PCT Filed: Apr. 6, 2021
(86) PCT No.: PCT/US2021/025939

§ 371 Date: Oct. 6, 2022

Related U.S. Application Data
(60) Provisional application No. 63/010,376, filed on Apr. 15, 2020.
(60) Provisional application No. 63/005,937, filed on Apr. 6, 2020.

(51) Int. Cl. A61K 31/352; A61P 11/00; A61K 9/51

(52) U.S. Cl. CPC A61K 31/352; A61P 11/00; A61K 9/5138

(57) ABSTRACT

Materials and methods for using genistein to treat respiratory distress syndrome or acute lung injury (e.g., pneumonitis, pulmonary fibrosis, dyspnea, pneumonia, and/or pulmonary edema resulting from viral infection, including SARS-CoV-2 infection) are provided herein.

### CROSS-REFERENCE TO RELATED APPLICATIONS

This application claims benefit of priority from U.S. Provisional Application Ser. No. 63/010,376, filed on Apr. 15, 2020, and U.S. Provisional Application Ser. No. 63/005,937, filed on Apr. 6, 2020. The disclosures of the prior applications are considered part of (and are incorporated by reference in) the disclosure of this application.

### TECHNICAL FIELD

This invention relates to materials and methods for reducing lung injuries such as pneumonitis, pneumonia, pulmonary fibrosis, and/or pulmonary edema in mammals with conditions such as coronavirus infections.

### BACKGROUND

Severe acute respiratory syndrome (SARS) is a viral respiratory illness caused by the SARS-associated coronavirus (SARS-CoV). SARS-CoV caused a global epidemic in 2002 and 2003, with more than 8000 confirmed cases in more than 25 countries. SARS-CoV-2 is a novel coronavirus that was first reported in December 2019 in Wuhan, China, and has resulted in a global pandemic with substantial morbidity and mortality (Zou et al., N Engl J Med 382:1177-1179, 2020).

### SUMMARY

Coronaviruses typically cause respiratory diseases. The novel SARS-CoV-2 coronavirus, which causes the disease known as COVID-19, is thought to rapidly invade and destroy human lung cells. The destroyed cells fill patients' airways with debris and fluid, leading to shortness of breath and triggering an immune response that results in an influx of cytokines, which can further exacerbate lung inflammation and respiratory distress. Thus, patients with severe cases of COVID-19 often present with severe pulmonary dysfunction, including shortness of breath, pulmonary edema, pneumonia, pneumonitis, and/or pulmonary fibrosis.

This application is based, at least in part, on the discovery that genistein can be used to treat mammals identified as having SARS-CoV-2. For example, genistein's ability to ameliorate pneumonitis and pulmonary fibrosis makes it useful as a therapeutic in COVID-19 patients, and particularly in patients suffering from the more severe, lung-related effects of the disease.

In one aspect, this document features a method for reducing pneumonitis, pulmonary fibrosis, dyspnea, pneumonia, and/or pulmonary edema in a mammal identified as being infected or having been infected with a coronavirus. The method can include, or consist essentially of, administering to the mammal a composition containing genistein in an amount effective to reduce pneumonitis, pulmonary fibrosis, dyspnea, pneumonia, and/or pulmonary edema in the mammal. The mammal can be a human. The mammal can be identified as being or having been infected with SARS-CoV-2. The composition can be formulated as a tablet, a capsule, a gel cap, or a powder. The genistein can be nanoparticulate genistein. The composition can have a nanoparticulate genistein concentration between about 250 mg/mL and about 500 mg/mL. The composition can contain nanoparticulate genistein with a particle size distribution characterized by a d(0.5) less than or equal to 0.5 p.m. The composition can further contain one or more pharmaceutically acceptable excipients forming a suspension medium, where the one or more pharmaceutically acceptable excipients include a water soluble polymer comprising a polyvinylpyrrolidone. The one or more pharmaceutically acceptable excipients can include a nonionic surfactant, a diluent, or a buffer. The composition can contain a nonionic surfactant in an amount ranging from about 0.01% to about 10% by weight (w/w). The amount of water soluble polymer in the composition can be about 0.5% to about 15% (w/w). The composition can contain a diluent and a preservative. The composition can further contain a non-ionic surfactant. The composition can contain nanoparticulate genistein in an amount ranging up to about 50% (w/w). The composition can contain nanoparticulate genistein in an amount of about 20% to about 35% (w/w). The composition can contain nanoparticulate genistein at a concentration of about 325 mg/mL. The composition can have a pH of about 2 to about 12. The genistein can be formulated as a solid dispersion. The method can include administering the composition orally, intramuscularly, subcutaneously, or intravenously. The method can include administering the composition within about 1 to about 96 hours of diagnosis with a coronavirus infection or within about 1 to 96 hours of onset of one or more symptoms of coronavirus infection. The method can include administering the composition beginning within about 1 hour to about 72 hours of the diagnosis of pneumonitis, pneumonia, or pulmonary fibrosis. The method can include administering the composition beginning about 4 to 8 weeks after diagnosis with a coronavirus infection or onset of one or more symptoms of coronavirus infection. The method can include administering the composition beginning about 8 to 12 weeks after diagnosis with a coronavirus infection or onset of one or more symptoms of coronavirus infection. The method can include administering the composition at least once daily. The method can include administering the composition in an amount of about 0.5 g to about 2.5 g. The method can include administering the composition in an amount of about 1 g to about 1.5 g.

In another aspect, this document features a method for reducing the development or likelihood of pneumonitis, pulmonary fibrosis, dyspnea, pneumonia, and/or pulmonary edema in a mammal identified as being at risk for infection with or exposure to a coronavirus. The method can include, or consist essentially of, administering to the mammal a composition containing genistein in an amount effective to reduce the development or likelihood of pneumonitis, pulmonary fibrosis, dyspnea, pneumonia, and/or pulmonary edema in the mammal. The mammal can be a human. The mammal can be identified as being at risk of infection with or exposure to SARS-CoV-2. The composition can be formulated as a tablet, a capsule, a gel cap, or a powder. The genistein can be nanoparticulate genistein. The composition can have a nanoparticulate genistein concentration between about 250 mg/mL and about 500 mg/mL. The composition can contain nanoparticulate genistein with a particle size distribution characterized by a d(0.5) less than or equal to 0.5 p.m. The composition can further contain one or more pharmaceutically acceptable excipients forming a suspension medium, where the one or more pharmaceutically acceptable excipients include a water soluble polymer comprising a polyvinylpyrrolidone. The one or more pharmaceutically acceptable excipients can include a nonionic surfactant, a diluent, or a buffer. The composition can contain a nonionic surfactant in an amount ranging from about 0.01% to about 10% by weight (w/w). The amount of water soluble polymer in the composition can be about 0.5% to about 15% (w/w). The composition can contain a diluent and a preservative. The composition can further contain a non-ionic surfactant. The composition can contain nanoparticulate genistein in an amount ranging up to about 50% (w/w). The composition can contain nanoparticulate genistein in an amount of about 20% to about 35% (w/w). The composition can contain nanoparticulate genistein at a concentration of about 325 mg/mL. The composition can have a pH of about 2 to about 12. The genistein can be formulated as a solid dispersion. The method can include administering the composition orally, intramuscularly, subcutaneously, or intravenously. The method can include administering the composition within about 1 to about 96 hours of potential exposure to a coronavirus or a subject having a coronavirus infection. The method can include administering the composition beginning within about 1 hour to about 72 hours of potential exposure to a coronavirus or a subject having a coronavirus infection. The method can include administering the composition at least once daily. The method can include administering the composition in an amount of about 0.5 g to about 2.5 g. The method can include administering the composition in an amount of about 1 g to about 1.5 g.

In another aspect, this document features the use of a composition containing genistein to reduce pneumonitis, pulmonary fibrosis, dyspnea, pneumonia, and/or pulmonary edema in a mammal identified as being infected or having been infected with a coronavirus. The mammal can be a human. The mammal can be identified as being or having been infected with SARS-CoV-2. The composition can be formulated as a tablet, a capsule, a gel cap, or a powder. The genistein can be nanoparticulate genistein. The composition can have a nanoparticulate genistein concentration between about 250 mg/mL and about 500 mg/mL. The composition can contain nanoparticulate genistein with a particle size distribution characterized by a d(0.5) less than or equal to 0.5 μm. The composition can further contain one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The composition can contain a nonionic surfactant in an amount ranging from about 0.01% to about 10% by weight (w/w). The amount of water soluble polymer can be about 0.5% to about 15% (w/w). The composition can contain a diluent and a preservative. The composition can further contain a non-ionic surfactant. The composition can contain nanoparticulate genistein in an amount ranging up to about 50% (w/w). The composition can contain nanoparticulate genistein in an amount of about 20% to about 35% (w/w). The composition can contain nanoparticulate genistein at a concentration of about 325 mg/mL. The composition can have a pH of about 2 to about 12. The genistein can be formulated as a solid dispersion. The composition can be formulated for oral, intramuscular, subcutaneous, or intravenous administration. The composition can be for administration within about 1 to about 96 hours of diagnosis with a coronavirus infection or within about 1 to 96 hours of onset of one or more symptoms of coronavirus infection. The composition can be for administration beginning within about 1 hour to about 72 hours of the diagnosis of pneumonitis, pneumonia, or pulmonary fibrosis. The composition can be for administration beginning about 4 to 8 weeks after diagnosis with a coronavirus infection or onset of one or more symptoms of coronavirus infection. The composition can be for administration beginning about 8 to 12 weeks after diagnosis with a coronavirus infection or onset of one or more symptoms of coronavirus infection. The composition can be for administration at least once daily. The composition can be formulated for administration in an amount of about 0.5 g to about 2.5 g. The composition can be formulated for administration in an amount of about 1 g to about 1.5 g.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C show that a composition containing an oral suspension of nanoparticulate genistein was able to mitigate the onset of radiation-induced edema and fibrosis in the lung. Mice were exposed to 11.0 Gy whole thoracic lung irradiation (WTLI) or sham irradiation and then treated with vehicle or genistein suspension (400 mg/kg) once daily for 2, three, or 4 weeks. FIG. 1A includes representative images of animal lungs stained with Masson's Trichrome. Images on the left are whole-lung, and images on the right are high magnification (10×). Unirradiated lungs showed well-aerated alveoli. Irradiated lungs of untreated mice displayed significant morphological changes, with almost complete loss of airways due to edema, congestion, and fibrotic scarring. The irradiated lungs of mice treated with genistein suspension (400 mg/kg) for 4 weeks showed significantly less tissue damage. For example, an absence of perivascular lymphocytic infiltration (arrow) was observed in treated mice. FIG. 1B is a graph plotting mean wet lung weight, showing that irradiated mice treated with genistein suspension had reduced mean wet lung weight as compared to animals that received radiation alone (p<0.05). Error bars ±SEM. FIG. 1C is a graph plotting the reduction in levels of fibrosis (determined by

Masson's Trichrome staining, with scoring as described by Ashcroft et al., J Clin Pathol 41:467-470, 1988) when genistein suspension was administered 24, 72, or 120 hours after radiation exposure.

FIGS. 2A-2C show that an oral suspension of nanoparticulate genistein mediated radioprotection of normal lung tissue in a mouse xenograft model. FIG. 2A is an image of a mouse in which A549 cells were implanted subcutaneously, followed by treatment with 12.5 Gy of radiotherapy (RT). Genistein suspension (200 mg/kg/d) was given by oral gavage beginning 7 days prior to whole thorax-lung irradiation (WTLI, RT), and continued daily until the end of the study. The white box indicates the path of radiation, and the circle indicates the tumor site. FIG. 2B contains representative lung tissue H&E images from the control (No RT), irradiated (RT), and genistein-treated (Genistein+RT) groups. FIG. 2C is a graph plotting wet weight of lungs from WTLI-treated animals after administration of vehicle or genistein, as an indicator of pneumonitis.

FIGS. 3A and 3B are graphs plotting pulmonary adverse event incidence rates in non-small cell lung cancer (NSCLC) clinical trials. The average incidence rate of dyspnea (FIG. 3A) and pulmonary fibrosis (FIG. 3B) in historical patients (Bradley et al., Lancet Oncol 16(2):187-199, 2015; solid bars; N=151) and genistein phase 1b/2a trial (white bars; N=21) are plotted.

FIGS. 4A-4F are graphs plotting serum levels of transforming growth factor beta (TGFβ isoform 1 (TGFβ1) (FIGS. 4A-4C) and TGFP isoform 2 (TGFβ2) (FIGS. 4D-4F) in NSCLC patients. Measurements were taken just prior to initiating genistein treatment (500 mg/day, FIGS. 4A and 4D; 1000 mg/day, FIGS. 4B and 4E; 1500 mg/day, FIGS. 4C and 4F), once weekly during concurrent genistein treatment and chemoradiotherapy (weeks one through six), once during consolidation, 3 months after completion of radiotherapy, and 6 months after completion of radiotherapy.

### DETAILED DESCRIPTION

Coronaviruses are RNA viruses that typically cause respiratory tract infections, sometimes lethally. For example, SARS-CoV and MERS-CoV are responsible for severe acute respiratory syndrome (SARS) and middle east respiratory syndrome (MERS), respectively, both of which can be deadly. The SARS-CoV-2 virus was first identified in patients exhibiting pneumonia and shortness of breath. SARS-CoV-2 enters host cells by binding the cellular receptor, angiotensin-converting enzyme 2 (ACE2), which is highly expressed in the epithelial cells of the airways and lungs (Jia et al., J Virol 79(23):14614-14621, 2005). Once SARS-CoV-2 infects cells, it triggers an immune response that involves recruitment of macrophages and neutrophils into the lungs, and the release of a pro-inflammatory cytokine storm. Cytokines that commonly are overexpressed in the serum of patients with SARS or COVID-19 include interleukin-6 (IL-6), interleukin-8 (IL-8), tumor necrosis factor alpha (TNFα), interferon gamma (INF-γ), and TGF (Dosch et al., Virus Res 142(1-2):19-27, 2009; Guo et al., Mil Med Res 7(1):11, 2020; and Lin et al., Emerg Microbes Infect 9(1):727-732, 2020). In serious cases of COVID-19, such as those that require hospitalization and mechanical ventilation, the severe immune response triggered in patients can result in acute respiratory distress syndrome (ARDS). In one study of 138 hospitalized patients with COVID-19, the median time until ARDS developed was eight days (Wang et al., JAMA 323(11):1061-1069, 2020). Infiltration of immune cells (particularly neutrophils) into the lungs is important because they produce factors that can inhibit viral replication and destroy infected cells to prevent further production of viral progeny (Galani and Andreakos, JLeukoc Biol 98(4):557-564, 2015). During ARDS, however, neutrophils and other immune cells contribute to lung injury by releasing toxic oxygen radicals and proteases that damage epithelial and endothelial cells, which results in increased alveolar permeability (Crimi and Slutsky, Best Pract Res Clin Anaesthesiol 18(3):477-492, 2004). In the acute phase of disease, this damage causes pulmonary edema and a potentially fatal decrease in respiratory function. In the later stage of disease, inflammation results in the over production of pro-fibrotic factors and collagen production, which leads to stiffening of the lungs and fibrosis. The pathophysiology of ARDS is not specific to SARS-CoV-2 infection, and is similar across other respiratory infections or pulmonary traumas (Reiss et al., Curr Opin Crit Care 24(1):1-9, 2018).

Pulmonary fibrosis due to ARDS is a primary reason that patients who recover from SARS have a lower quality of life and lower respiratory function (Wang et al., Zhonghua Shao Shang Za Zhi 36(0):E006, 2020, doi 10.3760/cma.j.cn501120-20200307-00132). Studies of patients with SARS showed that 45% had pulmonary fibrosis one month after infection, while 28% had pulmonary fibrosis twelve months after infection (Venkataraman and Frieman, Antiviral Res 143:142-150, 2017). Case studies of patients hospitalized with severe COVID-19 indicated that some developed pulmonary fibrosis as early as the first week of infection, and the percentage of patients with pulmonary fibrosis ballooned by the second or third week of infection (Zhang, Intensive Care Med 2020, doi 10.1007/s00134-020-05990-y; and Xiong et al., Invest Radio! 2020, doi 10.1097/RLI.0000000000000674). In addition, studies in a transgenic mouse that expresses human ACE2 in the lungs showed that the animals developed pneumonitis within three days of infection with SARS-CoV-2, and developed pulmonary fibrosis within five days of infection (Bao et al., bioRxiv 2020:2020.02.07.939389, doi 10.1101/2020.02.07.939389).

As described herein, compositions containing genistein can be used as medical countermeasures or therapeutics for reducing pneumonitis and/or pulmonary fibrosis in COVID-19 patients. Genistein [5,7-dihydroxy-3-(4-hydroxyphenyl)-4H-chromen-4-one (USAN); 5,7-dihydroxy-3-(4-hydroxyphenyl)-chromen-4-one (IUPAC); 5,7-dihydroxy-3-(4-hydroxyphenyl)-4H-1-benzopyran-4-one; 5,7,4′-trihydroxyisoflavone; or 4′,5,7-trihydroxyisoflavone) is a phytoestrogen in the category of isoflavones. Genistein's chemical structure is shown in Formula (1):

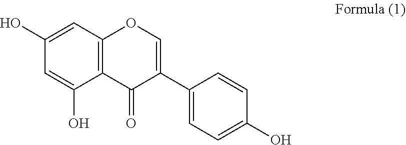 [SMILES: O=c1c(-c2ccc(O)cc2)coc2cc(O)cc(O)c12]

Genistein is one of several known isoflavones that are normally found in plants. The main sources of natural genistein are soybeans and other legumes. Genistein also is commercially available, and may be obtained in synthetic, purified form (e.g., from DSM Nutritional Products, Inc., Parsippany, N.J.).

Genistein has the ability to reduce expression of NF-κB, pro-inflammatory cytokines [e.g., IL-1, IL-6, and TNFα), and pro-fibrotic proteins (e.g., TGFβ, matrix metalloproteinase-2 (MMP-2), matrix metalloproteinase-9 (MMP-9) and lactate dehydrogenase (LDH)] (see, Day et al., J Rad Res 49(4):361-372, 2008; Ha et al., Rad Res 180(3):316-325, 2013; Ji et al., PloS One 7(12):e53101, 2012; Kang et al., Crit Care Med 31(2):517-524, 2003; Kang et al., Am J Resp Crit Care Med 164(12):2206-2212, 2001; Kim et al., Int Journal Mol Med 34(6):1669-1674, 2014; Morris et al., Rad Res 135(3):320-331, 1993; and Zhu et al., Biosci Biotechnol Biochem 84(3):544-551, 2020), all likely occurring via activation of estrogen receptor beta (ERβ) and downstream repression of NF-κB. Genistein's effects on NF-κB, pro-inflammatory cytokines, and pro-fibrotic proteins are evidence of its anti-fibrotic and anti-inflammatory properties. Data providing further evidence of genistein's effects are discussed in the Examples below. Thus, compositions containing genistein can be used to reduce or prevent pulmonary fibrosis, pneumonitis, and/or other adverse lung events (e.g., dyspnea, pulmonary edema, and/or pneumonia) in subjects identified as having, being at risk of having a coronavirus infection, including COVID-19, as being at increased risk of exposure a coronavirus.

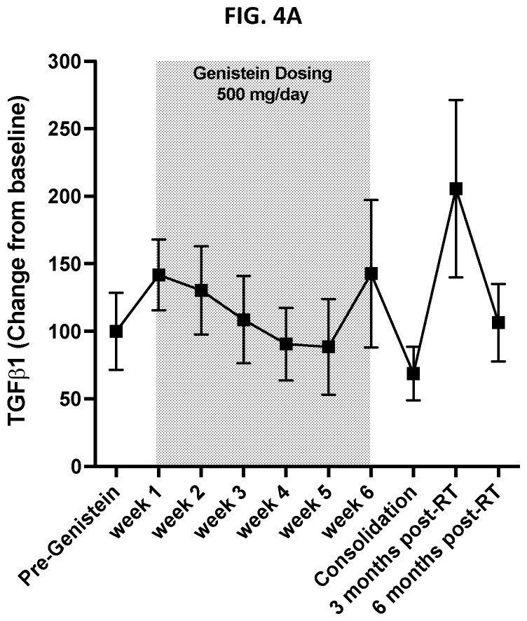

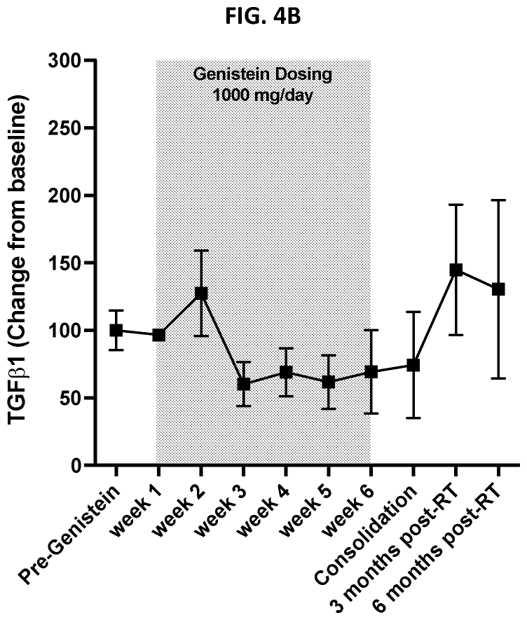

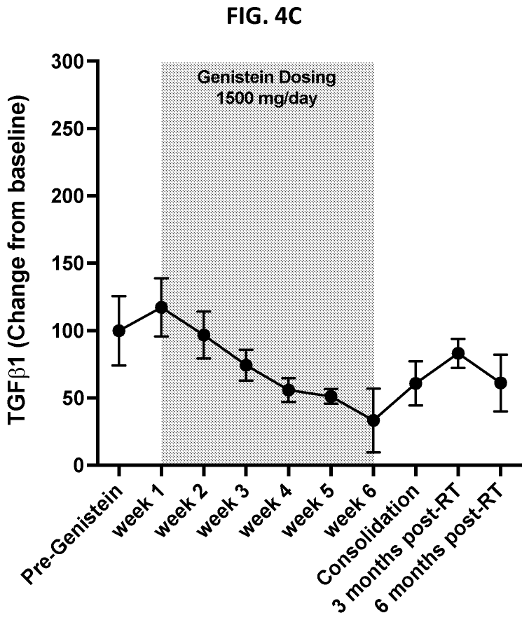

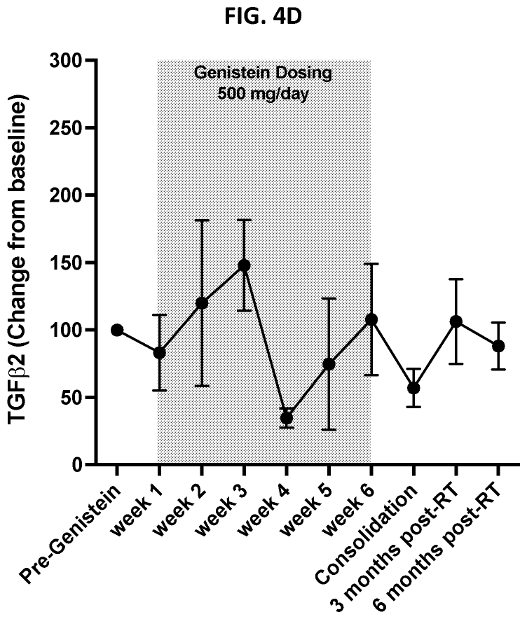

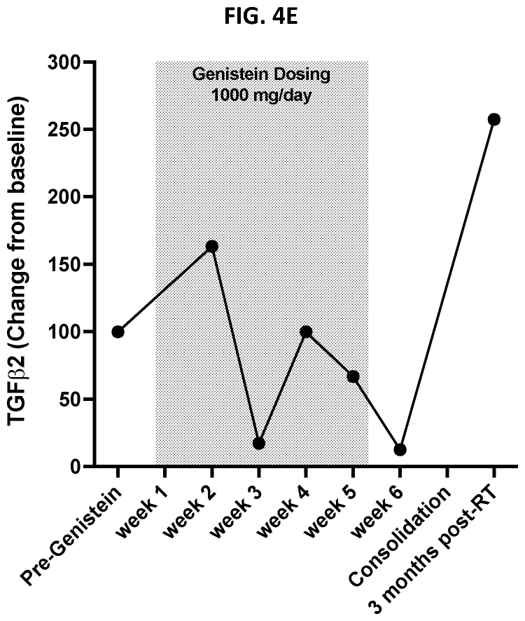

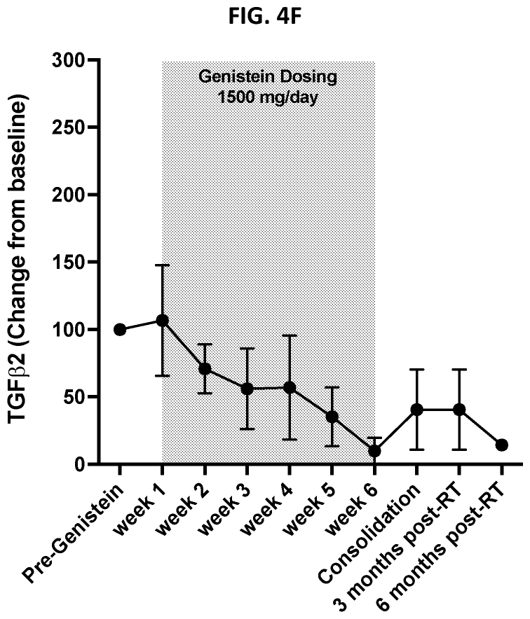

Any appropriate genistein-containing composition can be used to alleviate pneumonitis, pulmonary fibrosis, dyspnea, pneumonia, and/or pulmonary edema, as described herein. In some cases, for example, a composition can include genistein nanoparticles, which can have improved oral and/or parenteral bioavailability as compared to genistein that is not in nanoparticle form. Nanoparticle formulations can contain sub-micron size genistein particles, which can be manufactured using a wet nanomilling process that reduces genistein to a median particle size of less than 0.2 μm. See, e.g., U.S. Pat. No. 8,551,530. Pharmacokinetic experiments using such a genistein nanosuspension in mice demonstrated dramatically increased oral bioavailability as compared to formulations containing non-micronized genistein. See, FIGS. 4-7 of U.S. Pat. No. 8,551,530.

In some embodiments, a composition can contain genistein (e.g., nanoparticulate genistein or genistein that is not in nanoparticle form), at a concentration between about 100 mg/mL and about 500 mg/mL (e.g., about 100 mg/mL to about 400 mg/mL, about 150 mg/mL to about 350 mg/mL, about 200 mg/mL to about 400 mg/mL, about 250 mg/mL to about 350 mg/mL, about 275 mg/mL to about 325 mg/mL, about 300 mg/mL to about 450 mg/mL, or about 350 mg/mL to about 500 mg/mL). Compositions containing nanoparticulate genistein can have a particle size distribution characterized by a median diameter [d(0.5)] that is less than or equal to 0.5 μm (e.g., less than or equal to 0.4 μm, less than or equal to 0.3 μm, or less than or equal to 0.2 μm). The composition also can contain one or more other components, as described herein (e.g., one or more pharmaceutically acceptable excipients that form a suspension medium, such as a water soluble polymer, a nonionic surfactant, a diluent, or a buffer). In some embodiments, the suspension medium may be non-aqueous, such as edible lipids, oils, and fats from plant and animal sources (e.g., olive, corn, soy, marine, coconut, palm, palm kernel, cotton seed, peanut, safflower, sesame, sunflower, almond, cashew, macadamia, pecan, pine nut, walnut, lemon, orange, flax seed, and borage oils).

In some cases, the genistein compositions used in the methods provided herein can be formulations that include genistein in a solution along with one or more pharmaceutically acceptable carriers, excipients, and/or diluents. In some cases, the genistein-containing compositions used in the methods provided herein can be suspension formulations that include genistein (e.g., nanoparticulate genistein) suspended in a medium containing one or more pharmaceutically acceptable carriers, excipients, and/or diluents. Pharmaceutically acceptable carriers, excipients, and diluents suitable for therapeutic use include those described, for example, in Remington's Pharmaceutical Sciences, Maack Publishing Co. (A. R. Gennaro (ed.), 1985). In some cases, polyethylene glycol (PEG) can be used as a carrier in a composition that also contains genistein that is not in nanoparticle form.

In some cases, genistein compositions can include a suspension containing nanoparticulate genistein suspended in a non-aqueous medium, such as an edible plant or animal oil (e.g., olive oil, sunflower oil, corn oil, soy oil, marine oil, coconut oil, palm oil, palm kernel oil, cotton seed oil, safflower oil, sesame oil, peanut oil, almond oil, cashew oil, pecan oil, pine nut oil, macadamia oil, orange oil, flax seed oil, lemon oil, walnut oil, borage oils, fish oils, and dairy derived fats). See, e.g., U.S. Pat. No. 9,084,726.

Genistein compositions can, in some cases, include a suspension containing nanoparticulate genistein suspended in a medium including one or more water soluble polymers and one or more nonionic surfactants. See, e.g., U.S. Pat. No. 8,551,530. Nonionic surfactants can facilitate wetting and aid in preventing agglomeration of genistein particles (e.g., nanoparticulate genistein). Suitable nonionic surfactants include, without limitation, polysorbates, poloxamers, polyoxyethylene castor oil derivatives, bile salts, lecithin, 12-hydroxystearic acid-polyethylene glycol copolymer, and the like. In some embodiments, a genistein composition can include a nonionic surfactant selected from the group consisting of polysorbate 80 (TWEEN® 80), polysorbate 20 (TWEEN® 20), Poloxamer 188, and combinations thereof. In some cases, the total nonionic surfactant content in a genistein composition can range from about 0.01% to about 10% by weight (w/w) (e.g., about 0.2% to about 5% (w/w), about 0.2% to about 2% (w/w), about 0.2% to about 1% (w/w), about 0.2% to about 0.6% (w/w), and about 0.2% to about 0.8% (w/w).

Water soluble polymers can serve to enhance the viscosity of a suspension and/or to stabilize genistein particles (e.g., nanoparticulate genistein) against particle agglomeration or potential deleterious effects from other formulation components, for example. Water soluble polymers are pharmaceutically acceptable polymers that can be dissolved or dispersed in water. Suitable water soluble polymers include, without limitation, vegetable gums (e.g., alginates, pectin, guar gum, and xanthan gum), modified starches, polyvinylpyrrolidone (PVP), hypromellose (HPMC), methylcellulose, and other cellulose derivatives (e.g., sodium carboxymethylcellulose, hydroxypropylcellulose, and the like). In some cases, the genistein compositions described herein can include a poloxamer (e.g., Poloxamer 188) as a water soluble polymer. Poloxamer 188 is both a polymer and surfactant. The total water soluble polymer content in a genistein composition as provided herein can range from about 0.5% to about 15% (w/w) [e.g., about 1% to about 10% (w/w), about 10% to about 15% (w/w), about 12% to about 15% (w/w), about 1% to about 8% (w/w), and about 1% to about 5% (w/w)].

Carriers suitable for use in the genistein formulations described herein also include pharmaceutically acceptable aqueous carriers such as, sterile water, physiologically buffered saline, Hank's solution, and Ringer's solution. The formulations also can contain one or more buffers [e.g., one or more citrate buffers, phosphate buffers, tris(hydroxymethyl)aminomethane (TRIS) buffers, and/or borate buffers], to achieve a desired pH and osmolality. Injectable pharmaceutical formulations typically have a pH in the range of about 2 to about 12. In some embodiments, the genistein formulations provided herein can have a pH that falls in a range that more closely approximates physiologic pH (e.g., about 4 to about 8, or about 5 to about 7).

The genistein compositions used in the methods described herein also can, in some cases, include one or more diluents. Suitable diluents include those selected from, without limitation, pharmaceutically acceptable buffers, solvents, and surfactants.

In some cases, a genistein composition can include PVP (e.g., 5% PVP-K17) and polysorbate 80 (e.g., 0.2% polysorbate 80), as well as phosphate buffered saline (PBS, e.g., 50 nM PBS) and one or more chelating agents (e.g., ethylenediaminetetraacetic acid; EDTA). In some cases, for example, an oral formulation of a genistein composition can contain PVP (e.g., PVP-K25), polysorbate 80 (TWEEN® 80), and one or more preservatives (e.g., methyl paraben, propyl paraben, benzyl alcohol, or any combination thereof). In addition, a composition can include a diluent such as a sodium chloride solution.

When a composition contains nanoparticulate genistein, the particle size distribution of the genistein nanoparticles can be, for example, d(0.5)≤0.70 microns (e.g., d(0.5)≤0.60 microns, d(0.5)≤0.50 microns, d(0.5)≤0.40 microns, d(0.5)≤0.30 microns, or d(0.5)≤0.20 microns). See, e.g., U.S. Pat. No. 8,551,530. It is to be noted that genistein formulations characterized as suspensions can contain a measurable amount of genistein dissolved in the suspension medium, depending on the carrier(s), excipient(s), and diluent(s) included in the suspension medium. Genistein exhibits low to virtually no solubility in several pharmaceutically acceptable solvents, but some formulations of genistein (e.g., nanoparticulate suspensions of genistein) can provide a high concentration of genistein. For example, a suspension of nanoparticulate genistein can incorporate genistein in amounts ranging from about 100 mg/mL to about 500 mg/mL (e.g., ranges from about 100 mg/mL to about 400 mg/mL, about 150 mg/mL to about 350 mg/mL, about 200 mg/mL to about 400 mg/mL, about 250 mg/mL to about 350 mg/mL, about 275 mg/mL to about 325 mg/mL, about 300 mg/mL to about 450 mg/mL, or about 350 mg/mL to about 500 mg/mL, or amounts of about 100 mg/mL, about 150 mg/mL, about 200 mg/mL, about 250 mg/mL, about 275 mg/mL, about 300 mg/mL, about 325 mg/mL, about 350 mg/mL, about 375 mg/mL, about 400 mg/mL, about 450 mg/mL, or about 500 mg/mL). The relative amount of genistein included in such a suspension can be varied to yield a formulation having a desired total content of genistein. For example, a suspension formulation as described herein can include up to about 50% (w/w) genistein [e.g., about 50% (w/w), about 45% (w/w), about 40% (w/w), about 35% (w/w), about 30% (w/w), about 25% (w/w), about 20% (w/w), about 15% (w/w), about 10% (w/w), about 40% to about 50% (w/w), about 35% to about 45%, about 30% to about 40% (w/w), about 25% to about 35% (w/w), about 20% to about 30% (w/w), about 20% to about 35% (w/w), about 15% to about 35%, about 10% to about 30%, or about 10% to about 25%1. In some embodiments, nanoparticle genistein suspensions can provide increased bioavailability of genistein as compared to the bioavailability of genistein provided by solution formulations (e.g., solutions containing a pharmaceutically acceptable PEG solvent or containing larger sized genistein material). As described in U.S. Pat. No. 8,551,530, for example, the combination of high genistein loading and significantly increased bioavailability can provide advantages, such as facilitating administration of therapeutically effective amounts of genistein using much lower amounts of formulated drug substance, for example.

Genistein compositions can be formulated for administration by any suitable method, depending upon whether local or systemic treatment is desired and upon the area to be treated. For example, a genistein composition can be formulated for oral administration, parenteral administration (e.g., by subcutaneous, intrathecal, intraventricular, intramuscular, or intraperitoneal injection, or by intravenous drip), pulmonary administration (e.g., by inhalation or insufflation of powders or aerosols or a nebulized mist), or by a combination of routes such as oral and parenteral administration. Administration can be rapid (e.g., by injection) or can occur over a period of time (e.g., by slow infusion or administration of slow release formulations, such as from subcutaneous drug depots, slow short term intravenous injections, or slow release oral formulations).

Compositions and formulations for parenteral administration include, for example, sterile solutions (e.g., sterile aqueous solutions or suspensions) that also can contain buffers, diluents, and/or other suitable additives (e.g., penetration enhancers, carrier compounds and other pharmaceutically acceptable carriers). Compositions formulated for parenteral delivery can be manufactured according to standard methods to provide sterile compositions deliverable via, for example, intravenous injection or infusion, intravascular injection, subcutaneous injection, or intramuscular injection. A genistein formulation (e.g., a suspension of nanoparticulate genistein) can be prepared to have a viscosity suitable for the desired route of parenteral administration, and can be manufactured and packaged in any manner suited to the desired application, including, without limitation, as a formulation deliverable via intravenous injection or infusion, intravascular injection, subcutaneous injection, or intramuscular injection. In some embodiments, a formulation as described herein can be contained in one or more pre-filled syringes or auto-injectors prepared for administration of a given dose or range of doses of genistein.

Genistein compositions also can be formulated for oral administration. Compositions and formulations for oral administration include, for example, powders, granules, suspensions or solutions in water or non-aqueous media (e.g., suspensions of genistein nanoparticles in edible oil), capsules, gel caps, sachets, and tablets. In some cases, a genistein composition can be prepared as a liquid suspension that can be metered to deliver a desired dose, or can be incorporated into capsules (e.g., gelatin or soft capsules) suitable for delivery of liquid formulations. Formulations for oral administration also can be loaded into prefilled sachets or premetered dosing cups. In some cases, such genistein formulations also can include one or more pharmaceutically acceptable sweetening agents, preservatives, dyestuffs, flavorings, or any combination thereof. Genistein-containing compositions can be produced for oral administration in any suitable packaging. In some cases, for example, powdered genistein can be combined with one or more excipients and packaged into individual or bulk containers that can be provided to a subject, who then can combine the genistein formulation with a beverage (e.g., water) for drinking.

In some cases, a solid formulation for oral administration can be prepared by spray drying an aqueous formulation of genistein to generate a powder containing crystals or nanocrystals. In some cases, a solid formulation for oral administration can be prepared by spray drying a solution containing genistein and one or more organic solvents to generate an amorphous solid dispersion. In some cases, a solid formulation for oral administration can be prepared by hot melt extrusion of a solution containing genistein and a polymer. U.S. Provisional Application No. 63/092,838 (which is incorporated herein by reference in its entirety) provides an additional discussion of such solid formulations of genistein. For example, solid dispersion formulations can include genistein and one or more pharmaceutically acceptable excipients. In some cases, a solid dispersion formulation can include one or more additional components or additives including, without limitation, one or more fillers, preservatives, colorants, flavorants, sweeteners, dispersants, antistatic agents, glidants, other processing aides (e.g., plasticizers), and the like. The additives can be added during manufacture of the solid dispersion formulation, or during a post processing step after the solid dispersion formulation has been formed.

In some cases, genistein can be dispersed substantially uniformly throughout the matrix of the pharmaceutically acceptable excipient that forms the solid dispersion. The amount of genistein in the solid dispersion can vary. For example, a solid dispersion formulation can include genistein at a concentration of between about 25% and about 50% (w/w), between about 25% and about 45% (w/w), or between about 30% and about 40% (w/w).

Various types of pharmaceutically acceptable excipients can be included in a solid dispersion. In some cases, the pharmaceutically acceptable excipients can include one or more water soluble polymers. Water soluble polymers include pharmaceutically acceptable polymers that can be dissolved or dispersed in water.

Suitable water soluble polymers for use in a solid dispersion formulation can be selected from, for example, vegetable gums, such as alginates, pectin, guar gum, and xanthan gum, modified starches, polyvinylpyrrolidone (PVP), polyvinylpyrrolidone-co-vinylacetate (PVPVA), hypromellose (HPMC), methylcellulose, and other cellulose derivatives (e.g., sodium carboxymethylcellulose or hydroxypropylcellulose). In some cases, a solid dispersion formulation can include polyvinylpyrrolidone as a water soluble polymer.

In some cases, the total content of the one or more pharmaceutically acceptable excipients in a solid dispersion can range from about 50% to about 75% (w/w), from about 55% to about 75% (w/w), or from about 60% to about 70% (w/w).

For example, a solid dispersion can contain from about 50% to about 75% (w/w) or from about 60% to about 70% (w/w) of a water soluble polymer such as polyvinylpyrrolidone.

A solid dispersion formulation can optionally include one or more additional components or additives such as fillers, preservatives, colorants, flavorants, sweeteners, dispersants, antistatic agents, glidants, or other processing aides (e.g., plasticizers). In some cases, a solid dispersion formulation can include a non-nutritive sweetener such as, without limitation, sucralose, aspartame, saccharin, or stevia. Other non-nutritive or nutritive sweeteners (e.g., dextrose, fructose, and/or sucrose) also can be used. The additives can be added during the manufacture of a solid dispersion formulation, or during a post processing step after the solid dispersion formulation has been formed. For example, a sweetener can be added after a solid dispersion formulation has been formed.

The solid dispersion formulations can be formed in various ways. In some cases, a solid dispersion formulation can be formed using a spray drying technique. When employing spray drying techniques, a mixture of genistein, one or more pharmaceutically acceptable excipients (e.g., polyvinylpyrrolidone), and one or more optional additives can be solubilized in a solvent to form a solution or suspension. Exemplary solvents include, without limitation, organic solvents such as dimethyl sulfoxide (DMSO), dimethyl formamide (DMF), methanol, ethanol, acetone, to dichloromethane, and combinations thereof. The solution or suspension then can be spray dried, resulting in an amorphous solid dispersion of genistein dispersed in a matrix of the one or more pharmaceutically acceptable excipients (e.g., polyvinylpyrrolidone) and the one or more optional additives. The one or more optional additives can also be blended after the amorphous solid dispersion has been spray dried and formed. In some cases, the amorphous solid dispersion includes a substantially uniform distribution of genistein throughout the matrix.

The particle or particulate size of a spray dried amorphous solid dispersion can vary depending on the parameters of the spray drying technique. In some cases, the particle or particulate size of a spray dried amorphous solid dispersion can be from about 1 micron to about 1000 microns, or from about 10 microns to about 1000 microns. Larger or smaller particles or particulates also can be formed, such as particles or particulates from about 1 micron to about 10 microns, from about 1 micron to about 100 microns, from about 100 microns to about 300 microns, from about 300 microns to about 600 microns, or from about 600 microns to about 1000 microns in size. Other sizes also are contemplated.

In some cases, a solid dispersion formulation can be formed using an extrusion technique, such as a hot melt extrusion technique. When employing hot melt extrusion techniques, a mixture of genistein particles, the one or more pharmaceutically acceptable excipients (e.g., polyvinylpyrrolidone), and the one or more optional additives can be heated to a melt temperature of from about 140° C. to about 220° C., or from about 160° C. to about 200° C. As the mixture is heated, the genistein particles and/or the one or more pharmaceutically acceptable excipients are melted or otherwise softened to produce a blend of genistein dispersed in the one or more melted or softened pharmaceutically acceptable excipients and one or more optional additives. The blend can then be extruded to form an amorphous solid dispersion of genistein dispersed in a matrix of the one or more pharmaceutically acceptable excipients (e.g., polyvinylpyrrolidone) and one or more optional additives.

The one or more optional additives also can be blended after the amorphous solid dispersion has been extruded and formed. In some cases, a solid dispersion can include a substantially uniform distribution of genistein throughout the matrix. Further, in some cases, the melt temperature of the extrusion can be at least 50° C., at least 60° C., at least 70° C., at least 80° C., at least 90° C., at least 100° C., or at least 110° C. lower than the melting point of the genistein. In further cases, the melt temperature of the extrusion can be between about 50° C. and about 150° C., or between about 100° C. and about 130° C. lower than the melting point of the genistein.

An extruded solid dispersion can be in various forms. In some cases, an extruded solid dispersion can be in pellet or rod form. In some cases, an extruded solid dispersion can be milled into smaller amorphous solid dispersion particles or particulates. In some cases, an extruded solid dispersion can be milled into particles or particulates from about 1 micron to about 1000 microns, or from about 10 microns to about 1000 microns in size. Larger or smaller particles or particulates can also be formed, such as particles or particulates from about 1 micron to about 10 microns, from about 1 micron to about 100 microns, from about 100 microns to about 300 microns, from about 300 microns to about 600 microns, or from about 600 microns to about 1000 microns in size. Other sizes also are contemplated.

In some cases, a composition can be formulated for pulmonary administration in the form of a mist, such as via a nebulizer. A nebulizer is a device that typically uses oxygen, compressed air, or ultrasonic power to break up a solution or suspension into small aerosol droplets that can be directly inhaled from the mouthpiece of the device. A nebulizer can be powered mechanically (e.g., by a user's pumping action or actuation of a spring to increase and then quickly decrease the air pressure in a container holding the composition), in which cases a volatile liquid (e.g., alcohol) may be added to the composition to facilitate the increase in pressure. In some cases, a nebulizer can be powered electrically, using a vibrating mesh or a compressor, or an oscillator that generates a high frequency ultrasonic wave to cause mechanical vibration of a piezoelectric element.

Genistein compositions useful in the methods described herein can further include any pharmaceutically acceptable genistein salts, esters, or salts of such esters, or any other genistein compound which, upon administration to an animal such as a human, is capable of providing (directly or indirectly) biologically active genistein or an active metabolite or residue thereof. Accordingly, for example, provided herein are pharmaceutically acceptable salts of genistein, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of genistein (e.g., salts that retain the desired biological activity of genistein without imparting undesired toxicological effects). Examples of pharmaceutically acceptable salts may include, for example, salts formed with cations (e.g., sodium, potassium, calcium, or polyamines such as spermine), acid addition salts formed with inorganic acids (e.g., hydrochloric acid, hydrobromic acid, sulfuric acid, phosphoric acid, or nitric acid), and salts formed with organic acids (e.g., glucuronic acid, acetic acid, citric acid, oxalic acid, palmitic acid, or fumaric acid). Depending on the route of administration, for example, genistein may be sulfated or in glucuronic acid form.

Compositions also can include other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the genistein within the composition. The formulations can be sterilized if desired.

This document also provides methods for using genistein compositions as countermeasures against the pulmonary effects of coronavirus infection. Such countermeasures can be used to prevent, reduce, or mitigate effects such as pneumonitis, pulmonary fibrosis, damage to the pulmonary system, degenerative effects on lung tissue, and even death. It is noted that the methods described herein also can be used as countermeasures against other indications and sources of lung injury. Such indications can include any condition that causes ARDS. The mechanical cause of ARDS is fluid leaked from the smallest blood vessels in the lungs into the tiny air sacs where blood is oxygenated. Normally, a protective membrane keeps this fluid in the vessels, but severe illness or injury can cause damage to the membrane, leading to the fluid leakage of ARDS. Underlying causes of ARDS include, without limitation, sepsis, inhalation of harmful substances (e.g., high concentrations of smoke or chemical fumes), aspiration or near-drowning episodes, severe pneumonia, head or chest injury, accidents such as falls or car crashes, pancreatitis, massive blood transfusions, burns, drugs that cause pneumonitis (e.g., some antibiotics, chemotherapy drugs, medications that maintain regular heartbeat, and overdoses of aspirin), repeated exposure to molds and/or bacteria, exposure to feathers or bird excrement, radiation treatment of the chest (e.g., for breast or lung cancer) or the whole body (e.g., in preparation for a bone marrow transplant), granulocyte colony-stimulating factor (G-CSF)-related pulmonary toxicity (e.g., in patients treated with NEUPOGEN®), and pneumonia caused by pathogens such as pneumococci, influenza, viruses, malaria, and mycoplasmas. In some cases, this document provides methods for treating a subject (e.g., a mammal, such as a human, a non-human primate, a mouse, a rat, a sheep, a pig, or a dog) to prevent or reduce one or more effects of infection by a coronavirus (e.g., SARS-CoV-2). The methods can include identifying a subject as having a coronavirus infection (e.g., based on symptoms or molecular diagnostic test), as having been exposed to coronavirus, or as being at risk for exposure to coronavirus, and administering to the subject an amount of a genistein-containing composition effective to reduce or prevent adverse effects of the coronavirus on the lung (e.g., pneumonitis or fibrosis). Subjects at risk for exposure to coronavirus include, for example, family members or other individuals (e.g., health care workers and first responders) in contact with a person who may have a coronavirus infection, or in contact with a person who has been exposed to someone else with a coronavirus infection. Thus, in some cases, individuals can be treated with a genistein-containing composition prophylactically, before exposure to coronavirus has occurred, or after exposure but before the onset of symptoms indicative of infection. In some cases, individuals can be treated with a genistein-containing composition after they present with symptoms that are consistent with a coronavirus infection (e.g., COVID-19), or after they are diagnosed as having a coronavirus infection (e.g., COVID-19).

Common symptoms of COVID-19 include fever, cough, and/or shortness of breath (dyspnea), but other symptoms include, for example, fatigue, headache, chills, sore throat, lost sense of taste or smell, runny nose, body aches, and diarrhea.

In some cases, a subject can be an individual identified as having a coronavirus infection. Such subjects can be treated on an hourly, daily, or weekly basis after being identified as having a coronavirus infection. In some cases, a genistein composition can be administered within about four days or less (e.g., within about 96 hours, within about 72 to 96 hours, about 48 to 72 hours, about 24 to 48 hours, about 20 to 24 hours, about 18 to 20 hours, about 16 to 18 hours, about 12 to 16 hours, about 8 to 12 hours, about 6 to 8 hours, about 4 to 6 hours, about 2 to 4 hours, about 1 to 2 hours, or within about 60 minutes) after diagnosis or after onset of one or more symptoms. In some cases, a genistein composition can be administered starting four days or more after diagnosis or onset of symptoms. For example, a genistein composition can be administered beginning about four to seven days, seven to 14 days, two to four weeks, four to eight weeks, eight to 12 weeks, 12 to 16 weeks, 16 to 20 weeks, or more than 20 weeks after diagnosis or onset of symptoms. In some cases, a genistein composition can be administered to a patient who has been hospitalized and released or to a mammal who tested positive for COVID-19 and has recovered, in order to reduce or prevent long term effects (e.g., pulmonary fibrosis, reduced pulmonary function as the patient recuperates. Administration can continue on an hourly, daily, weekly, or monthly basis to mitigate the effects of the coronavirus. For example, a genistein-containing composition can be administered one or more times daily, every other day, biweekly, weekly, bimonthly, monthly, or less often, for any suitable length of time after infection by the virus has been identified (e.g., for about a week, about two weeks, about three weeks, about a month, about six weeks, about two months, about three months, about six months, about a year, or more than a year after diagnosis).

In some cases, a subject can be an individual exposed to a coronavirus (e.g., in a medical setting such as a clinic or a hospital, in another public setting, or in a non-public setting such as a household with a person having a coronavirus infection). Subjects exposed to coronavirus can be treated on an hourly, daily, or weekly basis after exposure, in order to mitigate harmful effects of exposure to the virus. In some embodiments, a genistein composition can be administered within about four days or less (e.g., within about 96 hours, within about 72 to 96 hours, about 48 to 72 hours, about 24 to 48 hours, about 20 to 24 hours, about 18 to 20 hours, about 16 to 18 hours, about 12 to 16 hours, about 8 to 12 hours, about 6 to 8 hours, about 4 to 6 hours, or about 2 to 4 hours), or within about 60 minutes or less (e.g., within about 45 to 60 minutes, about 30 to 45 minutes, about 15 to 30 minutes, about 10 to 15 minutes, or about 5 to 10 minutes) after exposure. Administration can continue on an hourly, daily, weekly, or monthly basis to mitigate the effects of exposure to the coronavirus. For example, a genistein-containing composition can be administered one or more times daily, every other day, biweekly, weekly, bimonthly, monthly, or less often, for any suitable length of time after exposure to the virus has occurred (e.g., for about a week, about two weeks, about three weeks, about a month, about six weeks, about two months, about three months, about six months, about a year, or more than a year after exposure). In some cases, an individual can be treated on a daily or weekly basis (e.g., every day, about six days per week, about five days per week, about four days per week, about three days per week, or about two days per week), before potential exposure to coronavirus. In some embodiments, an individual can be treated within about 1 hour to about 6 days (e.g., within about 5 to 6 days, about 4 to 5 days, about 3 to 4 days, about 60 to 72 hours, about 48 to 60 hours, about 36 to 48 hours, about 24 to 36 hours, about 18 to 24 hours, about 12 to 18 hours, about 10 to 12 hours, about 8 to 10 hours, about 6 to 8 hours, about 4 to 6 hours, about 2 to 4 hours, or about 1 to 2 hours) before potential exposure, such as before entering an area of increased risk for exposure to coronavirus (e.g., a clinical or hospital setting), in order to prevent or reduce potential harmful effects, should such exposure occur.

The methods provided herein include administering to a subject a composition that contains genistein in any formulation suitable to deliver an effective amount of the genistein to the subject, where the amount is effective to prevent or reduce one or more adverse pulmonary events (e.g., coronavirus-related pneumonitis and/or pulmonary fibrosis) in the subject, and/or to increase lung function in the subject. As used herein, an amount that is “effective to reduce” one or more adverse pulmonary effects of coronavirus in a subject is an amount that is sufficient to reduce one or more effects of infection (e.g., pneumonitis, pulmonary fibrosis, pneumonia, dyspnea, pulmonary edema) by any amount (e.g., at least 5%, at least 10%, at least 15%, at least 20%, or at least 25%), as compared to the effect of infection observed before administration of genistein or at an earlier time point during genistein treatment, or as compared to the level in a corresponding subject to whom genistein was not administered. The level of pneumonitis, pulmonary fibrosis, pneumonia, dyspnea, and pulmonary edema can be evaluated by, for example, one or more of computerized tomography (CT) scanning, spirometry, diffusing capacity for carbon monoxide (DLCO), and plethysmography.

Lung function can be assessed by, for example, spirometry or six-minute walk test. As used herein, an amount that is “effective to improve” or “effective to increase” lung function in a subject is an amount that is sufficient to increase lung function (e.g., as assessed by spirometry or six minute walk test) by any amount (e.g., at least 5%, at least 10%, at least 15%, at least 20%, or at least 25%), as compared to the level of lung function observed before administration of genistein or at an earlier time point during genistein treatment, or as compared to the level in a corresponding subject to whom genistein was not administered. For example, in some cases, an effective amount of a genistein composition can be an amount that results in an increase (e.g., an increase of at least 5%, at least 10%, at least 15%, at least 20%, or at least 25%) in distance walked during a set (e.g., six minute) time period, or an increase (e.g., an increase of at least 5%, at least 10%, at least 15%, at least 20%, or at least 25%) in the volume or amount of air that a subject can inhale and/or exhale. In some cases, an effective amount of a genistein composition can be an amount that results in a reduced need for exogenous oxygen in the patient (e.g., an amount that leads to a reduction of at least 5%, at least 10%, at least 15%, at least 20%, or at least 25% in the amount of exogenous oxygen required to maintain sufficient blood oxygenation in a patient). In some cases, an effective dose can prevent development of one or more adverse pulmonary effects of the coronavirus. Thus, therapeutically or prophylactically effective doses of a genistein composition can be effective to prevent, reduce, or mitigate effects of exposure to a coronavirus (e.g., SARS-CoV-2) that include, without limitation, pneumonitis, pulmonary fibrosis, pneumonia, dyspnea, pulmonary edema, and death.

In some embodiments, a therapeutic or prophylactic dose of a genistein-containing composition for administration to a human can contain about 0.25 g to about 5 g of genistein (e.g., about 0.25 g to about 0.3 g, about 0.3 g to about 0.4 g, about 0.4 g to about 0.5 g, about 0.5 g to about 0.75 g, about 0.75 g to about 1 g, about 1 g to about 1.25 g, about 1.25 g to about 1.5 g, about 1.5 g to about 1.75 g, about 1.75 g to about 2 g, about 2 g to about 2.25 g, about 2.25 g to about 2.5 g, about 2.5 g to about 3 g, about 3 g to about 4 g, or about 4 g to about 5 g). In some cases, an amount of a genistein-containing composition can be effective to achieve a blood concentration of aglycone (unconjugated, non-glucuronidated) genistein in a human of about 10 nM to about 10 μM (e.g., about 10 nM to about 25 nM, about 25 nM to about 50 nM, about 50 nM to about 75 nM, about 75 nM to about 100 nm, about 88 nM to about 880 nM, about 88 nM to about 150 nm, about 100 nM to about 200 nM, about 200 nM to about 500 nM, about 500 nM to about 800 nM, about 750 nM to about 1 μM, about 1 μM to about 2 μM, about 2 μM to about 5 μM, or about 5 μM to about 10 μM).

In some cases, a genistein composition can be administered to a subject (e.g., a human) at a dose of about 2.5 mg/kg to about 1 g/kg (e.g., about 2.5 to about 5 mg/kg, about 5 to 10 mg/kg, about 10 to 25 mg/kg, about 25 to 50 mg/kg, about 50 mg/kg, about 75 to 100 mg/kg, about 100 to 200 mg/kg, about 200 to 300 mg/kg, about 300 to 400 mg/kg, about 400 to 500 mg/kg, about 500 to about 750 mg/kg, or about 750 mg/kg to about 1 g/kg). The dose can be administered on a daily to weekly basis or longer (e.g., for about 1 day to about 2 days, such as from 1 to 3 days, 3 to 7 days, 7 to 10 days, 10 to 14 days, 14 to 21 days, 21 to 28 days, 28 to 35 days, 30 to 45 days, 45 to 60 days, two to three months, three to six months, six to nine months, or nine months to a year).

The administering step can be accomplished via any suitable route. In some embodiments, for example, a genistein composition containing a solution of genistein or a suspension of genistein nanoparticles can be administered orally or parenterally (e.g., by injection, such as subcutaneous, intravenous, or intramuscular injection).

In some embodiments, a therapeutic method can include administering a first dose of genistein for a first period of time after exposure (or potential exposure) to a coronavirus or after diagnosis with a coronavirus infection), and then administering a second dose of genistein for a second period of time. The first dose can be higher than the second dose. For example, a method can include administering a genistein-containing composition to a human at a dose of 0.25 g to about 5 g per day (e.g., about 0.25 g to about 0.3 g, about 0.3 g to about 0.4 g, about 0.4 g to about 0.5 g, about 0.5 g to about 0.75 g, about 0.75 g to about 1 g, about 1 g to about 1.25 g, about 1.25 g to about 1.5 g, about 1.5 g to about 1.75 g, about 1.75 g to about 2 g, about 2 g to about 2.25 g, about 2.25 g to about 2.5 g, about 2.5 g to about 3 g, about 3 g to about 4 g, or about 4 g to about 5 g per day) for about 1 day to about 2 weeks (e.g., about 1 day to about 3 days, about 3 days to about 7 days, about 7 days to about 10 days, or about 10 days to about 14 days), and then administering the composition at a dose of about 0.1 g to about 1 g per day (e.g., about 0.1 g to about 0.7 g, 0.2 g to about 0.5 g, about 0.3 g to about 1 g, about 0.5 g to about 0.8 g, or about 0.5 g to about 1 g) for about 1 day to about 2 months (e.g., about 1 to 3 days, about 3 to 7 days, about 7 to 10 days, about 10 to 14 days, about 14 to 21 days, about 21 to 28 days, about 28 to 35 days, about 30 to 45 days, about 45 to 60 days, or about two to three months).

If a subject fails to respond to a particular amount of genistein, then the amount of the administered genistein composition can be increased by, for example, two fold. After receiving this higher amount, the subject can be monitored for both responsiveness to the treatment and toxicity symptoms, and further adjustments can be made accordingly. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration of a genistein composition can be any frequency that reduces inflammatory pulmonary injury (e.g., pneumonitis or fibrosis) without producing significant toxicity to the subject. For example, the frequency of administration can be from about once an hour to about once a week (e.g., from about four times daily to twice daily, about twice daily to once daily, about four times a week to twice a week, or about twice a week to once a week). The frequency of administration can remain constant or can be variable during the duration of treatment. A course of treatment with a composition containing genistein can include rest periods. In some cases, a composition containing genistein can be administered daily over a week-long period, followed by a rest period of one to seven days, and such a regimen can be repeated multiple times. As with the effective amount, various factors can influence the actual frequency of administration used for a particular application. In some cases, the effective amount, duration of treatment, use of multiple treatment agents, route of administration, and severity of the condition may require an increase or decrease in administration frequency.

An effective duration for administering a composition containing genistein can be any duration that reduces inflammatory pulmonary injury (e.g., pneumonitis or fibrosis) without producing significant toxicity to the subject. In some cases, the effective duration can vary from several days to several months. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated can be monitored. Any appropriate method can be used to determine whether or not pulmonary inflammation in a subject has been reduced. For example, a subject can be assessed by CT scan, spirometry, DLCO, or plethysmography after administration of genistein to determine if the treatment reduced the amount of pneumonitis and/or pulmonary fibrosis in the subject, as compared to the level of pneumonitis and/or pulmonary fibrosis observed in the subject prior to treatment.

This document also provides for the use of genistein compositions as described herein for preventing, reducing, or mitigating one or more effects of infection by or exposure to a coronavirus (e.g., SARS-CoV-2) in a subject identified as having been infected by or exposed to the coronavirus, or as being at risk of exposure to the coronavirus. In addition, this document provides for the use of genistein in the manufacture of medicaments for preventing, reducing, or mitigating one or more effects of infection by or exposure to coronavirus in a subject identified as having a coronavirus infection, as having been exposed to coronavirus, or as being at risk of exposure to coronavirus.

In addition, genistein formulations (e.g., genistein solutions or nanoparticle suspensions) can be combined with packaging material and sold as kits for preventing, reducing, or mitigating the effects of infection by or exposure to coronavirus. Thus, this document also provides articles of manufacture that can include one or more genistein-containing compositions. The articles of manufacture can further include, for example, buffers or other control reagents for reducing, preventing, or monitoring the effects of exposure to coronavirus. Instructions describing how genistein formulations are effective for preventing, reducing, or mitigating damage from such exposure also can be included in such kits.

In some embodiments, an article of manufacture can include a genistein formulation (e.g., a suspension of nanoparticulate genistein) contained within a means for administration, such as an auto-injector or a nebulizer. For example, an auto-injector or nebulizer can contain a suspension of nanoparticulate genistein at a concentration between about 250 mg/mL and about 500 mg/mL, where the genistein nanoparticulate composition has a particle size distribution characterized by a d(0.5) of 0.5 μm or less (e.g., 0.4 μm or less, 0.3 μm or less, or 0.2 μm or less). The genistein composition also can include other components (e.g., one or more pharmaceutically acceptable excipients), as described herein. Components and methods for producing articles of manufacture include those known in the art, for example. In addition, in some embodiments, pre-made auto-injectors and nebulizers can be obtained commercially, filled with a genistein nanoparticulate composition, and packaged as a kit for treating the effects of respiratory distress syndromes (e.g., pneumonitis, pulmonary fibrosis, pneumonia, dyspnea, and/or pulmonary edema).

The invention will be further described in the following examples, which do not limit the scope of the invention described in the claims.

### EXAMPLES

### Example 1

### Efficacy of Genistein for reducing Lung Fibrosis

Human lung tissue is known to undergo extensive degenerative remodeling following acute radiation exposure. This pathology, referred to as Delayed Effects of Acute Radiation Exposure in lung (DEARE-lung), results in shortness of breath, fatigue, and even death, all of which are caused by pneumonitis and pulmonary fibrosis. Analogous effects are observed in lung cancer patients receiving radiotherapy, because the radiation exposure can damage surrounding healthy tissue in addition to the tumor tissue. Mechanistically, the pathophysiology of DEARE-lung is similar to virally induced ARDS, in the sense that damage to the lung epithelium and endothelium results in an excessive immune response that further compounds tissue damage.

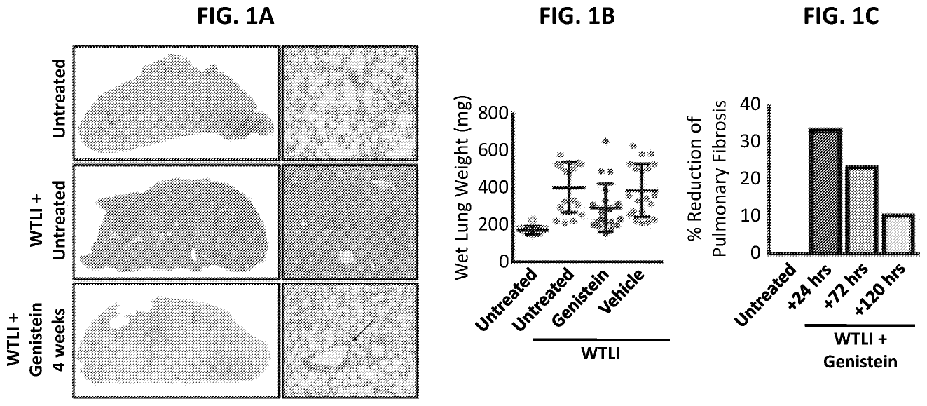

Studies using an oral suspension of nanoparticulate genistein demonstrated efficacy in a mouse model of DEARE-lung. In these studies, mice received a single dose of whole thoracic lung irradiation (WTLI), followed by daily treatment with the genistein composition beginning 24 hours after irradiation. Animals were treated for two, four, or six weeks. As compared to controls, the animals treated with genistein had significantly less immune infiltrate, edema, and fibrosis of the lungs as determined by histopathology and wet lung weight (FIGS. 1A and 1B). A reduction in pulmonary fibrosis was observed when genistein treatment started within five days of radiation, but the sooner treatment started, the better the results (FIG. 1C).

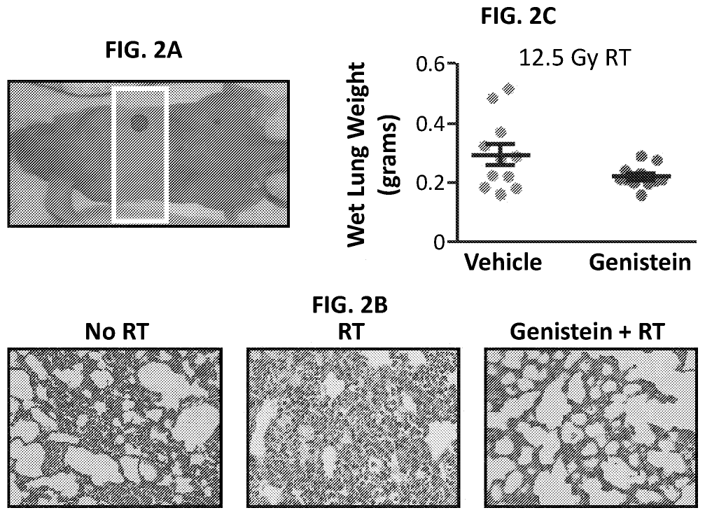

Other nonclinical studies focused on protection of the lungs in the context of cancer radiotherapy. A549 lung cancer cells were implanted subcutaneously in athymic CD1 nu/nu mice. The location of the implant was specific, so that a single dose of WTLI would hit the tumor and the normal lung tissue (FIG. 2A). Mice were then administered a nanoparticulate genistein suspension or vehicle control by daily oral gavage for seven days prior to WTLI and continuing until the end of the study. These studies showed that mice treated with genistein had a marked reduction in normal lung tissue remodeling (reduced thickening of the septal wall), as well as reduced congestion and minimal cellular infiltrate, as indicated by thin tissue sectioning and staining with hematoxylin and eosin (FIG. 2B). In addition, a reduction of pneumonitis was confirmed by reduced wet lung weight in genistein-treated animals, compared to animals treated with vehicle (FIG. 2C).

The use of genistein oral suspension also was studied in NSCLC patients. The drug was added to patients' chemoradiotherapy regimens to reduce the incidence of therapy-induced pneumonitis and pulmonary fibrosis. In particular, patients were given oral syringes preloaded with genistein oral suspension, so they could self-administer the drug at home. Patients ingested the suspension once daily for one week prior to chemoradiotherapy, and also while on concurrent chemoradiotherapy for a total of six to eight weeks. No dose-limiting toxicities were observed in any of the three dosing cohorts (500 mg/day, 1000 mg/day and 1500 mg/day).

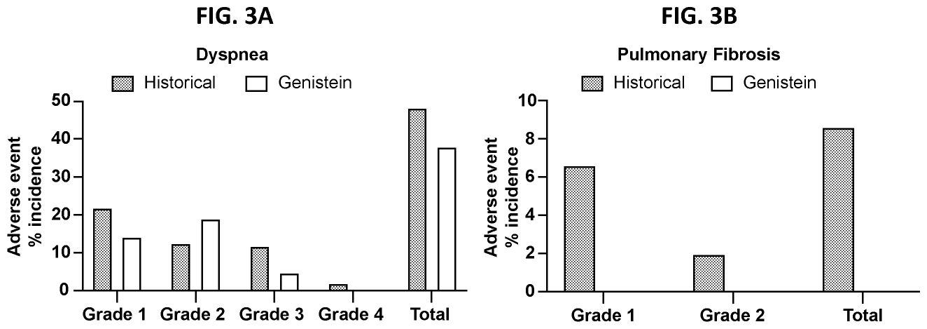

Pharmacokinetic evaluation demonstrated that all three dose levels produced serum levels of (biologically active) genistein aglycone that were sufficient to activate ERβ, although even at the highest dose (1500 mg/day), Cₘₐₓ levels were not adequate to reach the predicted EC50 for estrogen receptor alpha (ERα). This was consistent with studies showing that genistein preferentially binds to ERβ over ERα (Landauer et al., J Rad Res 60(3):308-317, 2019). Interim data analysis was compared to a historical trial (Bradley et al., supra) that used the same chemoradiotherapy regimen but without genistein. The analysis showed that patients had a lower incidence of hematological, pulmonary, and gastrointestinal adverse events when treated with the nanoparticulate genistein oral suspension, and that such incidences were less severe in the genistein-treated patients. This included dyspnea, which was less severe in genistein-treated patients (FIG. 3A), and pulmonary fibrosis, which 9% of patients in the historical study developed, but which none of the 21 patients treated with genistein have developed to date (FIG. 3B).

In further studies, serum levels of the cytokines TGFβ isoform 1 (TGFβ1) and

TGFβ isoform 2 (TGFβ2) were measured in patients as markers of lung damage and wound healing. Measurements were taken just prior to initiating genistein treatment, once weekly during concurrent genistein treatment and chemoradiotherapy (weeks one through six), once during consolidation, 3 months after completion of radiotherapy, and 6 months after completion of radiotherapy. Subjects that had pre-genistein cytokine measurements and at least 4 measurements during concurrent genistein and chemoradiotherapy treatment were included in the analysis (TGFβ1, N=5-6 and TGFβ2, N=1-4). The analysis revealed a dose-dependent decrease in cytokine levels for both TGFβ1 (FIGS. 4A-4C) and TGFβ2 (FIGS. 4D-4F) while patients were in the genistein dosing phase. TGFβ1 and TGFβ2 levels reverted to near baseline levels at the conclusion of genistein dosing, specifically in the lower dosing cohorts (500 mg and 1000 mg). These studies indicated that circulating levels of

TGFβ1 and TGFβ2 were reduced in a genistein dose-dependent manner while the patients were in the genistein dosing phase (weeks one through six) and receiving chemoradiotherapy, which resulted in no occurrences of pulmonary fibrosis in these patients one year later.

### Example 2

### Effectiveness of Genistein in SARS-CoV Models

Several animal models have been used to study SARS-CoV infection; these include ferrets, nonhuman primates (commonly African green monkeys), hamsters, and mice. SARS-CoV produces large viral titers in ferrets, but there is conflicting evidence that infection in ferrets demonstrates clinical symptoms of lung injury, so these models are thought to be best used for vaccine development (Venkataraman and Frieman, supra; and Gretebeck et al., Curr Opin Virol 13:123-129, 2015). Nonhuman primates produce similar clinical signs and viral titers as humans. In addition, a variety of mouse models exist for SARS-CoV. The most promising mouse model that is applicable to SARS-CoV-2 research is the K18-hACE2 transgenic mouse (McCray et al., J Virol 81(2):813-821, 2007). This transgenic model uses the K18 promoter to drive expression of human ACE2, which is the host receptor through which SARS-CoV and SARS-CoV-2 gain entry into cells. Studies using this model for SARS research demonstrated that it produces a pathology similar to that observed in humans, and that it involves substantial pulmonary injury (Dediego et al., Virol 376(2):379-389, 2008; and McCray, supra). Another study using a transgenic mouse expressing human ACE2 showed infection-induced weight loss and also showed signs of pneumonia and pulmonary fibrosis, which were detected within 3-5 days post infection (Bao et al., supra). Thus, it is anticipated that the mouse model also produces a SARS-CoV-2 pathology similar to that observed in humans.

Given the above, the mouse model is used to test the efficacy of genistein for preventing lung injury due to SARS-CoV-2 infection. Without being bound by a particular mechanism, it is believed that genistein treatment can prevent infection-induced pneumonia and pulmonary fibrosis by inhibiting the virus-induced inflammatory response, leading to increased survival and decreased weight loss in the animals.

Mice are treated according to the schedule in TABLE 1:

TABLE 1
K18-hACE2 Mouse Study 1

| | Test Article | Dose Level | | | No. of |
|---|---|---|---|---|---|
| Group | Treatment | (mg/kg/dose) | Route | Schedule | Animals |
| 1 | Vehicle | — | Oral | BID, 7 d | 15-20 |
| 2 | Genistein | 100 | Oral | BID, 7 d | 15-20 |
| 3 | Genistein | 200 | Oral | BID, 7 d | 15-20 |
| 4 | Genistein | 400 | Oral | BID, 7 d | 15-20 |
| 5 | Vehicle | — | Oral | BID, 6 d | 15-20 |
| 6 | Genistein | 200 | Oral | BID, 6 d | 15-20 |

Two mouse studies are used to evaluate genistein's efficacy against COVID-19. Study 1 is conducted to quickly identify signs of efficacy and determine the optimal dose level. In groups 1-4, mice are challenged with a molecular clone of SARS-CoV-2 through intranasal inoculation and then randomized into one of four treatment groups (TABLE 1). In groups 5 and 6, mice are treated with genistein prior to being challenged with a molecular clone of SARS-CoV-2 through intranasal inoculation. Based on previous SARS studies with this model, lung injury is likely to occur in vehicle-treated mice within three to five days post infection (dpi), and mice are likely to succumb to disease in seven to ten days dpi. Mice in groups 1-4 are treated with genistein or vehicle starting two hours after infection, as suggested by previous studies in a rat model of radiation-induced erectile dysfunction (RiED), which demonstrated that genistein treatment starting two hours after radiation exposure significantly reduced fibrosis. In the clinic, patients would presumably be treated at the onset of symptoms, but since disease progresses significantly faster in mice than in humans, treatment in mice is initiated shortly after infection. Genistein or vehicle is administered by oral gavage twice daily (BID) within about two hours after infection, and administration is continued for 7 days. Animals in groups 5 and 6 are administered genistein or vehicle by oral gavage BID for 6 days prior to infection. Animals in all groups that survive for 14 days are euthanized for histopathological analysis. The genistein dose levels in groups 3 and 6 are based on levels that showed efficacy in previous studies (FIGS. 1A-1C and 2A-2C). Additionally, dose levels 100 mg/kg and 200 mg/kg administered BID are roughly equivalent to the human doses 1000 mg and 2000 mg according to the FDA Center for Drug Evaluation and Research guidance for conversion of animal doses to human equivalent doses based on body surface area. In addition, the Group 4 dose level is included because the radiation-induced fibrosis efficacy studies involved at least four weeks of dosing, and since disease progresses so quickly in this model, a two-fold higher dose was included. Primary endpoints for this experiment are survival and body weight, which are monitored daily. This allows for expeditious completion and analysis of the study results so that the second mouse study design can be finalized and initiated. Next, signs of lung injury are examined following established protocols (Jackson et al., Int J Rad Oncol Biol Phys 105(2):400-409, 2019; and Jackson et al., Brit J Pharmacol 174(24):4738-4750, 2017) by removing lungs from euthanized animals and measuring wet lung weight as a sign of pneumonitis. Lungs then are fixed and sectioned for histopathological analysis by H&E and Masson Trichrome stain to look for histopathological signs of injury and collagen buildup, respectively. It is anticipated that treatment with genistein prevents pulmonary injury in this model, leading to an increase in survival.

For a second mouse study, animals are treated according to TABLE 2:

TABLE 2
K18-hACE2 Mouse Study 2

| | Test Article | Dose Level | | | No. of |
|---|---|---|---|---|---|
| Group | Treatment | (mg/kg/dose) | Route | Schedule | Animals |
| 1 | Vehicle | — | Oral | BID, 7 d | 20-25 |
| 2 | Genistein | 100-400 | Oral | BID, 7 d | 20-25 |

In the second study, the same mouse model is utilized for a natural history study that is performed to support positive results from the first study and to characterize the anti-inflammatory effects of genistein in SARS-CoV-2 infected mice (TABLE 2). The study is designed to assess the benefit of genistein on organs of interest (e.g., the heart, kidney, spleen, and/or brain), and to determine the impact of genistein on viral replication. The optimal dose level that produces positive results in Study 1 is repeated.

Mice are treated with genistein or vehicle starting two hours after infection as described above, continuing BID for 7 days post-infection. On days 3, 5, and 7, up to 6 mice are euthanized so that disease progression can be monitored in genistein or vehicle treated animals. All remaining mice that survive 14 days are euthanized for histopathological analysis. Survival and body weight are monitored to recapitulate previous findings. Disease progression is monitored over time by histopathological analysis in up to 6 different tissues for all euthanized mice. Further, terminal blood is collected at each time point in order to analyze expression of serum cytokines; complete blood counts with white blood cell differential also are obtained. Finally, RNA is extracted from tissues of the lung and trachea and nasal turbinate. RNA is used to detect genomic and sub-genomic SARS-CoV-2 RNA as a measure of viral load and replication. RNA also is used for transcriptomics in order to identify biomarkers of genistein bioactivity. These experiments demonstrate efficacy by improved lung pathology and reduced pro-inflammatory cytokine expression.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-26. (canceled)

27. A method for reducing pneumonitis, pulmonary fibrosis, dyspnea, pneumonia, and/or pulmonary edema in a mammal identified as being infected or having been infected with a coronavirus, the method comprising administering to the mammal a composition comprising genistein in an amount effective to reduce pneumonitis, pulmonary fibrosis, dyspnea, pneumonia, and/or pulmonary edema in the mammal.

28. The method of claim 27, wherein the mammal is a human.

29. The method of claim 27, wherein the mammal is identified as being or having been infected with SARS-CoV-2.

30. (canceled)

31. The method of claim 27, wherein the genistein is nanoparticulate genistein.

32. The method of claim 31, wherein the composition has a nanoparticulate genistein concentration between about 250 mg/mL and about 500 mg/mL.

33. The method of claim 31, wherein the composition comprises nanoparticulate genistein with a particle size distribution characterized by a d(0.5) less than or equal to 0.5 μm.

34. The method of claim 31, wherein the composition further comprises one or more pharmaceutically acceptable excipients forming a suspension medium, wherein the one or more pharmaceutically acceptable excipients include a water soluble polymer comprising a polyvinylpyrrolidone.

35. The method of claim 34, wherein the one or more pharmaceutically acceptable excipients include a nonionic surfactant, a diluent, or a buffer.

36-37. (canceled)

38. The method of claim 31, wherein the composition comprises a diluent and a preservative.

39. The method of claim 38, wherein the composition further comprises a non-ionic surfactant.

40-41. (canceled)

42. The method of claim 31, wherein the composition comprises nanoparticulate genistein at a concentration of about 325 mg/mL.

43-44. (canceled)

45. The method of claim 27, comprising administering the composition orally, intramuscularly, subcutaneously, or intravenously.

46. The method of claim 27, comprising administering the composition within about 1 to about 96 hours of diagnosis with a coronavirus infection or within about 1 to 96 hours of onset of one or more symptoms of coronavirus infection.

47. The method of claim 27, comprising administering the composition beginning within about 1 hour to about 72 hours of the diagnosis of pneumonitis, pneumonia, or pulmonary fibrosis.

48. The method of claim 27, comprising administering the composition beginning about 4 to 8 weeks after diagnosis with a coronavirus infection or onset of one or more symptoms of coronavirus infection.

49. The method of claim 27, comprising administering the composition beginning about 8 to 12 weeks after diagnosis with a coronavirus infection or onset of one or more symptoms of coronavirus infection.

50. The method of claim 27, comprising administering the composition at least once daily.

51. The method of claim 27, comprising administering the composition in an amount of about 0.5 g to about 2.5 g.

52. The method of claim 27, comprising administering the composition in an amount of about 1 g to about 1.5 g.

53. The method of claim 27, comprising administering the composition beginning more than 20 weeks after diagnosis with a coronavirus infection or onset of one or more symptoms of coronavirus infection.
